(19) United States
(12) Patent Application Publication
(10) Pub. No.: US 2026/0252993 A1
(43) Pub. Date: Aug. 27, 2026

(54) AI AGENT CONTROL OF COMPUTER EXECUTED WORKFLOWS IN RESPONSE TO GENERATIVE AI DIALOGUE WITH A USER

(71) Applicant: Iatrion Inc., San Francisco, CA (US)

(72) Inventor: Shahnaz Miri, San Francisco, CA (US)

(21) Appl. No.: 19/546,838

(22) Filed: Feb. 23, 2026

Related U.S. Application Data
(60) Provisional application No. 63/762,024, filed on Feb. 23, 2025.

(51) Int. Cl. G06Q 10/0631; G06F 16/3329

(52) U.S. Cl. CPC G06Q 10/06316; G06F 16/33295

(57) ABSTRACT

Methods, systems, and apparatus, including computer programs encoded on computer storage media for artificial intelligence (AI) agent control of computer executed workflows in response to generative AI dialogue with a user. Multiple workflow nodes are defined including questions to be answered by a user and/or required data to be received from a user. An artificial intelligence (AI) agent having a session memory map and a dynamic workflow controller (DWC) executes one or more of the workflow nodes. The AI agent generates dialogue with a user based on the parameters of a workflow node. During the dialogue with the user, the AI agent stores in the session memory map one or more inputs received from the user. The AI agent performs an operation to validate the output generated by one or more large language models (LLMs). Also, the AI Agent performs red-flag sub-workflow nodes based on the received one or more inputs from the user.

### CROSS-REFERENCE TO RELATED APPLICATIONS

This application is a non-provisional application of U.S. Provisional Application No. 63/762,024, filed on Feb. 23, 2025, which is hereby incorporated by reference in its entirety.

### FIELD

This application relates generally to control of computer executed workflows, and more particularly, to systems and methods for artificial intelligence (AI) agent control of computer executed workflows in response to generative AI dialogue with a user including, in some embodiments, clinical and healthcare-related data collection and documentation workflows.

### BACKGROUND OF THE INVENTION

Efficient and accurate collection of patient history is critical for optimal clinical workflow, yet traditional methods—such as manual or static intake forms—often result in incomplete data and increased administrative burden. While certain AI-powered solutions exist for post-visit transcription and general symptom inquiry, none adequately addresses the need for specialty-specific, adaptive pre-visit symptom collection that aligns with current clinical guidelines and ICD-10 standards.

Conventional systems also typically lack adaptive, medical specialty-specific questioning. Conventional systems do not dynamically adjust the interrogation process in real time based on patient responses or integrate comprehensive clinical guidelines. Most existing solutions rely on a single mode of communication rather than integrating dual communication modalities such as both traditional telephony and web-based voice interactions. Also, adequate encryption for cloud-based data storage and rigorous patient authentication protocols are generally inadequate in many conventional systems.

Thus, there is a pressing need for an integrated system that not only automates comprehensive patient history collection but also ensures data security, patient authentication, and structured clinical documentation while strictly serving as a decision support tool and remaining applicable to other regulated data collection environments.

### SUMMARY

In some embodiments, methods, systems, and apparatus, including computer programs encoded on computer storage media are described for artificial intelligence (AI) agent control of computer executed workflows in response to generative AI dialogue with a user. The described workflows may be applied to regulated data collection environments, including healthcare, without limitation. Multiple workflow nodes are defined including questions to be answered by a user and/or required data to be received from a user. An artificial intelligence (AI) agent having a session memory map and a dynamic workflow controller (DWC) executes one or more of the workflow nodes. The AI agent generates dialogue with a user based on the parameters of a workflow node. During the dialogue with the user, the AI agent stores in the session memory map one or more inputs received from the user. The AI agent performs an operation to validate the output generated by one or more large language models (LLMs). Also, the AI Agent performs red-flag sub-workflow nodes based on the received one or more inputs from the user.

The system relates to artificial intelligence (AI)-enabled medical documentation systems, and more specifically to methods and systems for automated pre-visit patient history collection, adaptive specialty-specific questioning, secure bidirectional telephony, and structured clinical documentation. The invention in certain embodiments is configured for is configured for collecting patient-reported symptoms and history, and for providing enhanced documentation and preliminary clinician's decision support based at least in part on such patient-reported information. In some implementations, the system may operate without requiring direct analysis of physiological measurements or diagnostic test results. In certain embodiments, the system functions as an assistive documentation and information-acquisition platform rather than an autonomous diagnostic or treatment-generating system.\

In some embodiments, the system provides an enhanced pre-visit workflow that automates comprehensive patient history collection, reducing clinician workload and streamlining clinical preparation.

In some embodiments, the system provides dual communication modalities that supports both outbound and inbound voice interactions with robust patient authentication, ensuring broad patient accessibility.

In some embodiments, the system provides adaptive, specialty-specific inquiries using a prompt-engineered LLM based on current guidelines to deliver focused, dynamic questioning that collects symptom characteristics relevant to clinician review of conditions subtypes.

In some embodiments, the system provides structured documentation and decision support. The system generates standardized clinical notes formatted for EHR integration and provides preliminary, differential diagnosis lists to assist clinical review without replacing clinical judgment. The differential outputs are generated for clinician-facing review and are not presented as diagnostic determinations.

In some embodiments, the system provides patient-accessible history summary for improved consultation readiness. The system provides patients with a structured version of their medical history summary in patient-friendly language, allowing them to better prepare for their medical consultations, particularly when their provider does not use this system. This ensures that patients can effectively communicate relevant symptoms and history to their healthcare provider, leading to more efficient and informed medical evaluations.

In some embodiments, the system provides secure data handling. The system incorporates encryption for all cloud-based data storage and implements rigorous authentication protocols for voice interactions, safeguarding patient information.

In some embodiments, the system is configured as a documentation and data-collection aid and is not configured to autonomously generate diagnoses or treatment recommendations.

The appended claims may serve as a summary of this application.

### BRIEF DESCRIPTION OF THE DRAWINGS

FIG. 1A is a diagram illustrating an exemplary environment in which some embodiments may operate.

FIG. 1B is a diagram illustrating an exemplary computer system with software and/or hardware modules that may execute some of the functionality described herein.

FIG. 2 is a block diagram illustrating different system layers.

FIG. 3 is a diagram illustrating a functional data flow of the system.

FIG. 4 is a diagram illustrating operations of the dynamic workflow controller (DWC).

FIG. 5 is a diagram illustrating a session state machine of the system.

FIG. 6 is a diagram illustrating a node processing flowchart.

FIG. 7 is a diagram illustrating a red-flag processing flowchart.

FIG. 8 is a diagram illustrating template mapping and factuality/provenance gate.

FIG. 9 is a diagram illustrating a clinician co-authoring merge processing flowchart.

FIG. 10 is a diagram illustrating a differential diagnosis (DDx) assist processing flow chart.

FIG. 11 is a diagram illustrating an admin editor/configuration layer of the system.

FIG. 12 is a diagram illustrating security/consent/retention control layers of the system.

FIG. 13 is a diagram illustrating a multi-specialty overlay.

FIG. 14 is a diagram illustrating exemplary data schemas for a workflow node and a session memory map.

FIG. 15 is a diagram illustrating next node selection and anti-duplication of workflow nodes.

FIG. 16 is a diagram illustrating an example transition table of a session state machine.

FIG. 17 is a diagram illustrating a provenance/factuality gate.

FIG. 18 is a flow chart illustrating an exemplary method that may be performed in some embodiments.

FIG. 19 is a diagram illustrating an exemplary computer system that may perform processing in some embodiments.

### DETAILED DESCRIPTION OF THE DRAWINGS

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

The present invention is an AI-driven medical assistant system designed to automate pre-visit patient history collection through adaptive, specialty-specific questioning, secure bidirectional voice communication, and structured clinical documentation generation. Key features of the invention include:

In some embodiments, the system provides functionality for adaptive LLM-based specialty-specific questioning. The system leverages a large language model (LLM) that is prompt-engineered using current medical literature, clinical guidelines, and ICD-10 standards. For example, in a neurology context, if the primary patient reported a complaint is headache, the AI initiates targeted questioning regarding headache frequency, quality, severity, duration, and associated symptoms. It asks questions regarding the quality and pattern of headaches and associated symptoms to structure symptom data relevant to clinically recognized headache categories based on established neurology guidelines (e.g., migraine type, tension type headache, cluster headache, or headaches related to increased intracranial hypertension) and specifically probes (with specific questions) for red flags such as associated vision decline, double vision, or nocturnal headaches. Additional queries capture the patient's known medical history and current medications. Patient responses dynamically refine the question flow to ensure complete and relevant data collection.

In some embodiments, the system provides bidirectional telephony and web-based voice calls. The system provides both outbound calls (scheduled pre-visit notifications) and inbound calls (initiated by patients or caregivers) through traditional telephony and web-based voice applications. Robust authentication protocols (e.g., using patient-specific identifying codes or date of birth, etc.) verify patient identity prior to initiating the adaptive questioning process.

In some embodiments, the system provides secure cloud-based data storage. All patient data, including transcribed text and optionally recorded audio, are securely stored in the cloud using strong encryption. This ensures compliance with data protection regulations and safeguards patient privacy.

In some embodiments, the system provides structured clinical documentation and decision support functionality. Upon completion of the adaptive interrogation, the system automatically generates a structured specialty-customized clinical note—comprising sections such as History of Present Illness (HPI), Review of Systems (ROS), medication lists, and relevant past medical history—formatted according to standardized protocols (e.g., HL7, FHIR) for optional seamless integration with electronic health records (EHRs). The system also provides a preliminary, non-diagnostic list of potential differential diagnoses based on the collection of patient's reported symptoms to support physician review.

In some embodiments, the system addresses may be configured for use in documentation, intake, and clinician-support workflows. The invention in certain implementations is configured for the collection of patient-reported symptoms and history taking, and for assisting with documentation and preliminary clinician's decision support. It may operate primarily on conversational and patient-reported information and function as an assistive documentation and workflow-support platform rather than an autonomous diagnostic or treatment-generating system.

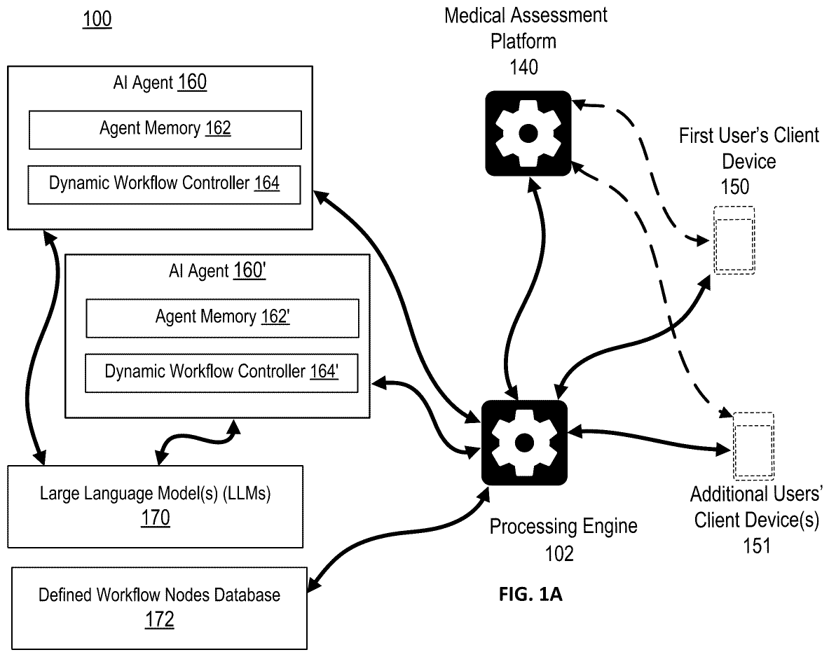

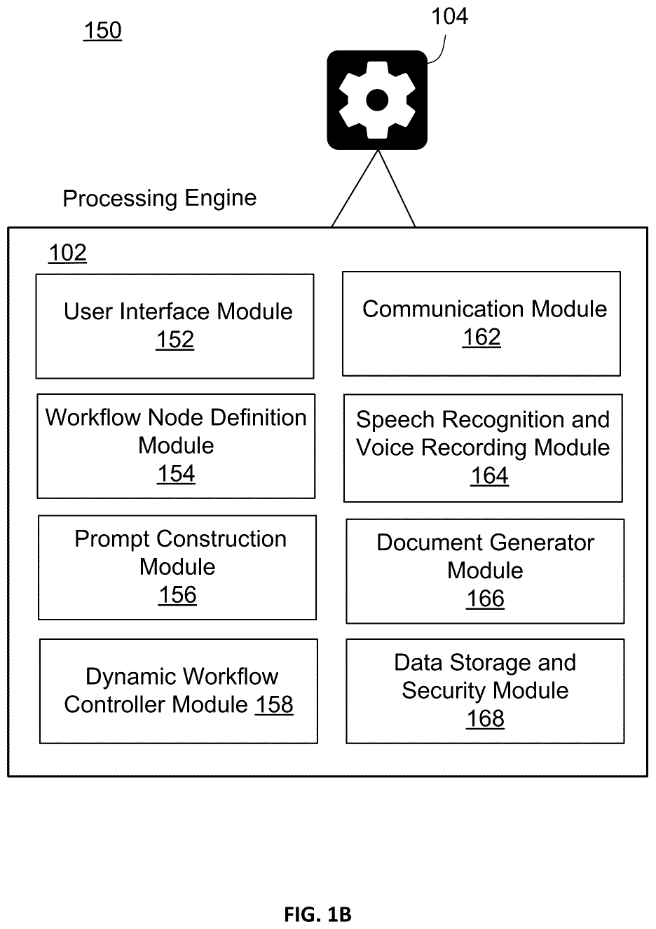

FIG. 1A is a diagram illustrating an exemplary environment in which some embodiments may operate. In the exemplary system 100, a first user's client device 150 and one or more additional users' client device(s) 151 are connected to a processing engine 102 and medical assessment platform 140. The processing engine 102 is connected to, and/or integrated with the medical assessment platform 140, and connected to one or more repositories (e.g., non-transitory data storage) and/or databases, including a Defined Workflow Nodes database 148. The first user's client device 150 and additional users' client device(s) 151 in this environment may be computers, and the medical assessment platform 140 and processing engine 102 may be applications or software hosted on a computer or multiple computers which are communicatively coupled via a remote server or via one or more local servers.

The exemplary system 100 is illustrated with only one additional user's client device, one processing engine, and medical assessment platform 140, though in practice there may be more or fewer additional users' client devices, processing engines, and/or document management platforms 140. In some embodiments, one or more of the first user's client device, additional users' client devices, processing engine, and/or medical assessment platform 140 may be part of the same computer or device. While the medical assessment platform 140 is illustrated as a type of software system that may be configured by the processes described herein, other types of systems, software or applications that may use different computing devices and/or peripherals may also be configured.

In an embodiment, the processing engine 102 may perform the methods described herein and, as a result, provide for a system and methods for artificial intelligence (AI) agent control of computer executed workflows in response to generative AI dialogue with a user. In some embodiments, this may be accomplished via communication with the first user's client device 150, additional users' client device(s) 151, processing engine 102, medical assessment platform 140, and/or other device(s) over a network between the device(s) and an application server or some other network server. In some embodiments, the processing engine 102 is an application, browser extension, or other piece of software hosted on a computer or similar device or is itself a computer or similar device configured to host an application, browser extension, or other piece of software to perform some of the methods and embodiments herein.

The first user's client device 150 and additional users' client device(s) 151 may be devices with a display configured to present information to a user of the device. In some embodiments, the first user's client device 150 and additional users' client device(s) 151 present information in the form of a user interface (UI) with UI elements or components. In some embodiments, the first user's client device 150 and additional users' client device(s) 151 send and receive signals and/or information to the processing engine 102 and/or medical assessment platform 140. The first user's client device 150 may be configured to perform functions related to document querying and/or document editing or redlining.

In some embodiments, the processing engine 102 is executed, in whole or part, on a server, on a respective client device, or a combination thereof. In some embodiments, the first user's client device 150 and additional users' client device(s) 151 are computing devices capable of hosting and executing one or more applications or other programs capable of sending and/or receiving information. In some embodiments, the first user's client device 150 and/or additional users' client device(s) 151 may be a computer desktop or laptop, mobile phone, video phone, or any other suitable computing device capable of sending and receiving information. In some embodiments, the processing engine 102 and/or medical assessment platform 140 may be hosted, in whole or in part, as an application or web service executed on the first user's client device 150 and/or additional users' client device(s) 151. In some embodiments, one or more of the medical assessment platform 140, processing engine 102, and first user's client device 150 or additional users' client devices 151 may be the same device. In some embodiments, the first user's client device 150 is associated with a first user account, and the additional users' client devices 151 accounts are associated with other user accounts on the medical assessment platform 140, and the additional users' client device(s) 151 are associated with additional user account(s) on the medical assessment platform 140.

The system 100 instantiates a respective AI Agent 160, 160′ for communication with a respective user, such as a first user and a second user. The AI Agent 160, 160′ interacts with one or more LLMs 170 to execute prompts based on requirements set forth in a workflow node. Workflow nodes may be defined and stored in a defined workflow nodes database 172. An AI agent may have an agent memory 162 where the agent maintains a session memory and/or state with information and/or data stored from the dialogue with a user. The AI agent includes a dynamic workflow controller 164 that dynamically controls a workflow and executes computer processes in response received user inputs.

FIG. 1B is a diagram illustrating an exemplary computer system 150 with software and/or hardware modules that may execute some of the functionality described herein. Computer system 150 may comprise, for example, a server or client device or a combination of server and client devices for automated configuration of software systems using images of hardware components or peripherals.

The User Interface Module 152 provides system functionality for presenting a user interface to one or more users of the medical assessment platform 140 and receiving and processing user input from the users. User inputs received by the user interface herein may include clicks, keyboard inputs, touch inputs, taps, swipes, gestures, voice commands, activation of interface controls, and other user inputs. In some embodiments, the User Interface Module 152 presents a visual user interface on a display screen. In some embodiments, the user interface may comprise audio user interfaces such as sound-based interfaces and voice commands.

The Workflow Node Definition Module 154 provides system functionality for defining and storing workflow nodes processed by the DWC 164, 164′. The DWC retrieves a particular defined workflow node and processes the node. As a result of processing the workflow node, the DWC dynamically executes one or more computer processes. In some embodiments, after completing a first workflow node, the DWC will process another workflow node. The selection of subsequent workflow nodes to perform may be deterministic (such as a predefined order of workflow nodes to perform) and/or or dynamic where the selection of the workflow nodes is based on some received input from a user

The Prompt Construction Module 156 provides system functionality to assembly prompt for use in execution by one or more large language models (LLMs). The AI agent 160, 160′ generates a prompt based on requirements set forth in a workflow node. The AI Agent submits the generated prompt to one or more LLMs 170 and receives an output from the LLM. The AI Agent then transmits a form of the received output to a user client device 150, 151.

The Dynamic Workflow Controller Module 158 (shown as Dynamic Workflow Controller 164, 164′) provides system functionality for dynamically controlling and executing one or more computer processes. The module 158 provides an LLM-Based adaptive question flow controller which receives user input and determines processes to be executed by the system based on the user input.

The Communication Module 162 provides system function for communication with other systems or computing device via one or more communication interfaces. In some embodiments, module 162 uses commercially available APIs (e.g., Twilio, WebRTC, SIP-based systems), the interface supports both outbound and inbound voice calls. Authentication protocols require patients to verify their identity using pre-assigned codes, date of birth, or similar identifiers before proceeding. The communication interface may provide communication protocols, such as: PSTN/WebRTC voice+DTMF; ASR for speech-to-text; optional TTS; patient authentication (DOB/code).

The Speech Recognition and Voice Recording Module 164 provides system functionality for converting speech-to-text and text-to-speech. The module 164 converts patient speech to text in real time and optionally records audio for verification and auditing. All captured data are encrypted and stored securely in a cloud environment.

The Documentation Generator Module 166 provides system functionality for generating information and documentation regarding patient collected information. Once patient data collection is complete, the system compiles the information into a structured clinical note. This note, which follows a specialty-specific template, includes the HPI, ROS, medication and allergy lists, and a preliminary non-diagnostic differential diagnosis list. The note is formatted for direct integration with EHRs using standard protocols (e.g., HL7, FHIR).

The Data Storage and Security Module 168 provides system functionality for data storage and system security. Patient data and audio recordings are stored on cloud servers with robust encryption. Access is restricted to authorized personnel, ensuring compliance with health data security regulations.

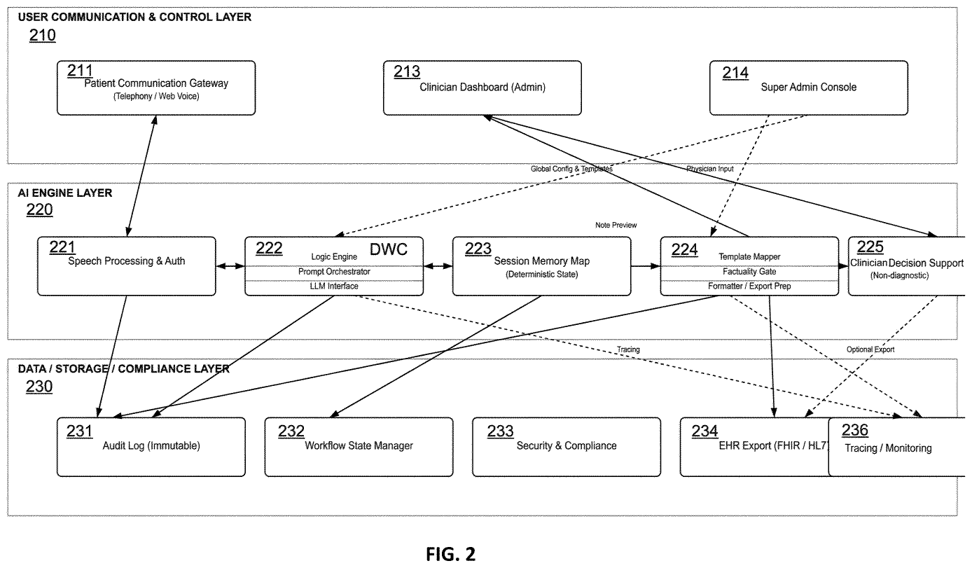

FIG. 2 is a block diagram illustrating different system layers. The block diagram illustrates the system in a layered architecture. The diagram exemplifies an AI-driven clinical documentation and decision-support system including user communication, AI-engine, and data/compliance layers. A User Communication Layer 210 comprises a Patient Communication Gateway 211, Clinician Dashboard 213, Super Admin Console 214.

An AI Engine Layer 220 comprises Speech Processing and Auth 221, including an ASR/TTS, Dynamic Workflow Controller (DWC) 222, Session Memory Map 223, Documentation Engine 224, Decision Support 225.

A Documentation Engine 224 shows internal subcomponents (Template Mapper, Factuality Gate, Formatter/Export Prep). The Engine 224 connects directly to Decision Support 225 and to EHR Export 234. Decision Support 225 also connects to EHR Export 234 (dashed “Optional Export”).

Tracing/Monitoring may be performed by the Dynamic Workflow Controller 222 and/or Documentation Engine 224 level. Also feed a separate Tracing/Monitoring Service 236 (dashed lines) and the Audit Log 231 (solid lines). This supports reproducibility and a feedback optimization loop without naming vendors.

Clinician Dashboard 213 displays the Note Preview from 224 and sends Physician Input into 225. Super Admin console 214 configures both DWC 222 and Documentation Engine 224 (dashed “Global Config & Templates”).

The Data/Compliance Layer 230 includes Audit Log 231, Workflow State Manager 232, Security & Compliance 233, EHR Export 234, and Tracing/Monitoring 236.

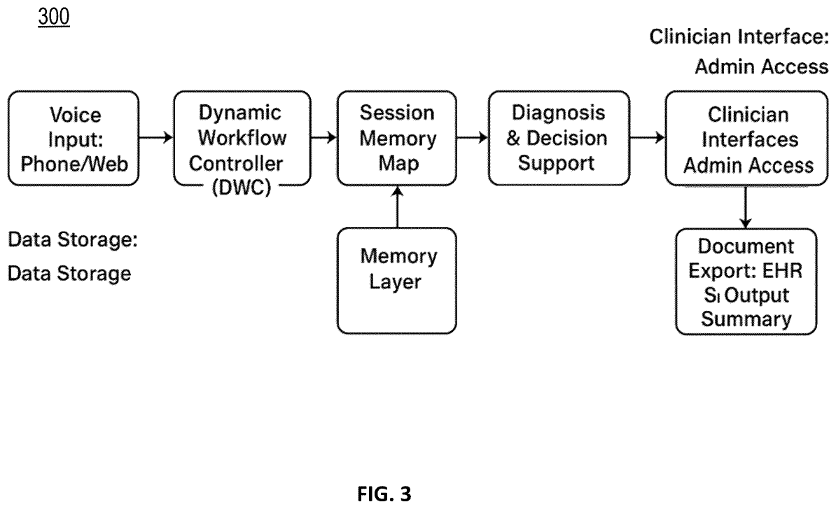

FIG. 3 is a diagram illustrating a functional data flow 300 of the system. The data flow of the system shows data and control flow in a chronological order. The flow of the system moves from Voice/ASR to DWC with Validators and Red-Flag Engine; Memory layer comprising Session Map, State DB, LLM Buffer, and Audit Log; Documentation Generator; Clinician Co-Authoring Interface; Decision-Support Panel; Export/EHR Adapter. Corresponding data types at each stage include Audio→Text→Structured JSON→Note→Final Output.

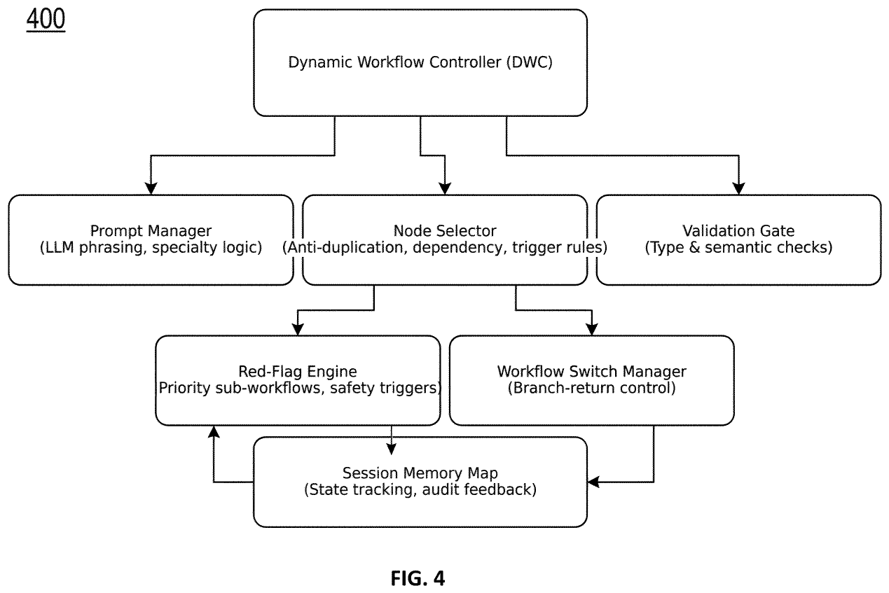

FIG. 4 is a diagram illustrating operations of the dynamic workflow controller (DWC). The DWC logic diagram 400 illustrates operation processing and rule-based controls of the DWC. Submodules performed by the DWC include a prompt manager, node selector, validation gate, red-flag engine, and workflow switch manager. Feedback and data are stored by the DWC in a session memory map (which allows state tracking and audit feedback).

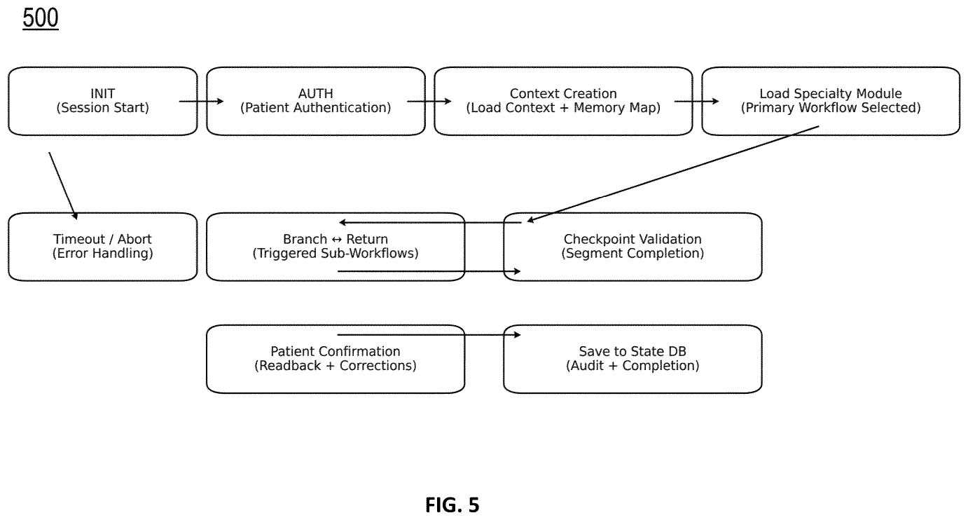

FIG. 5 is a diagram illustrating a session state machine 500 of the system. The diagram illustrates how a conversational state evolves with a user. The states comprises: INIT→AUTH→Context Creation→Load Specialty Module→Branch↔Return↔Checkpoint→Patient Confirmation→Save to State DB. The session state machine provides a deterministic conversation flow, branching, checkpoints and error handling.

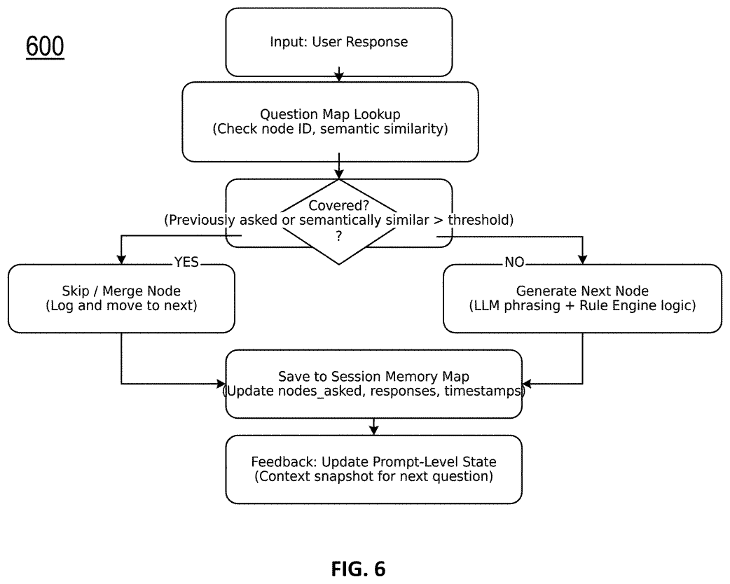

FIG. 6 is a diagram illustrating a node processing flowchart. The process 600 for workflow node processing and selection is described and illustrates the avoidance of repetitive questioning. Input: user_response→Question Map Lookup→(if covered?)→Skip/Merge→Else→Generate Next Node (LLM+Rules)→Save to Session Memory. This processing performed by the system 100 provides efficient and non-redundant operations.

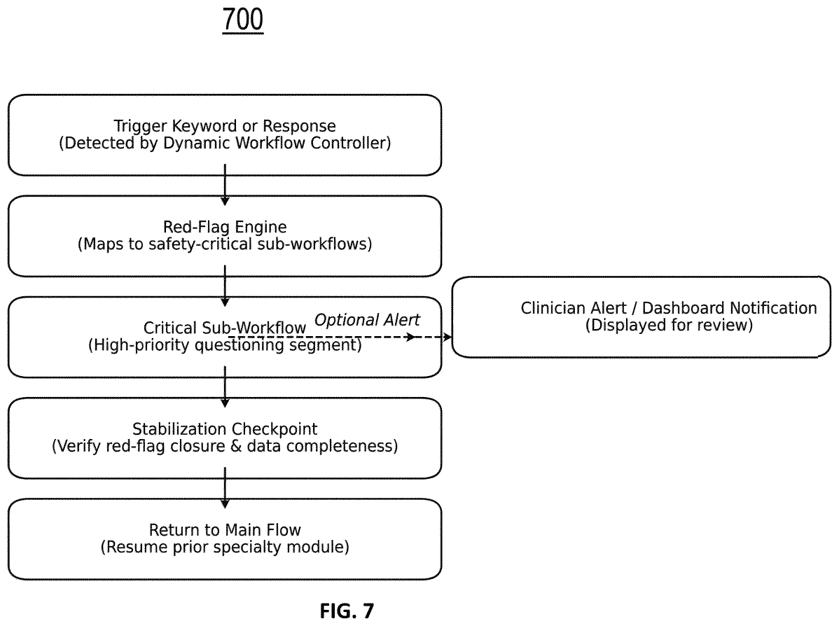

FIG. 7 is a diagram illustrating a red-flag processing flowchart. The process 700 for red-flag branch flow provides a safety mechanism distinct from ordinary questioning. The process is initiated from Trigger keyword→Red-Flag Engine→Critical Sub-Workflow→Stabilization Checkpoint→Return to Main Flow. The processing flow provides safety and compliance assurance. In some embodiments, the red-flag branch flow processing is initiated by the system 100 as a safety-critical sub-workflow and clinical alert path wherein the red-flag sub-workflow generates informational indicators for clinician review without issuing diagnoses or treatment directives.

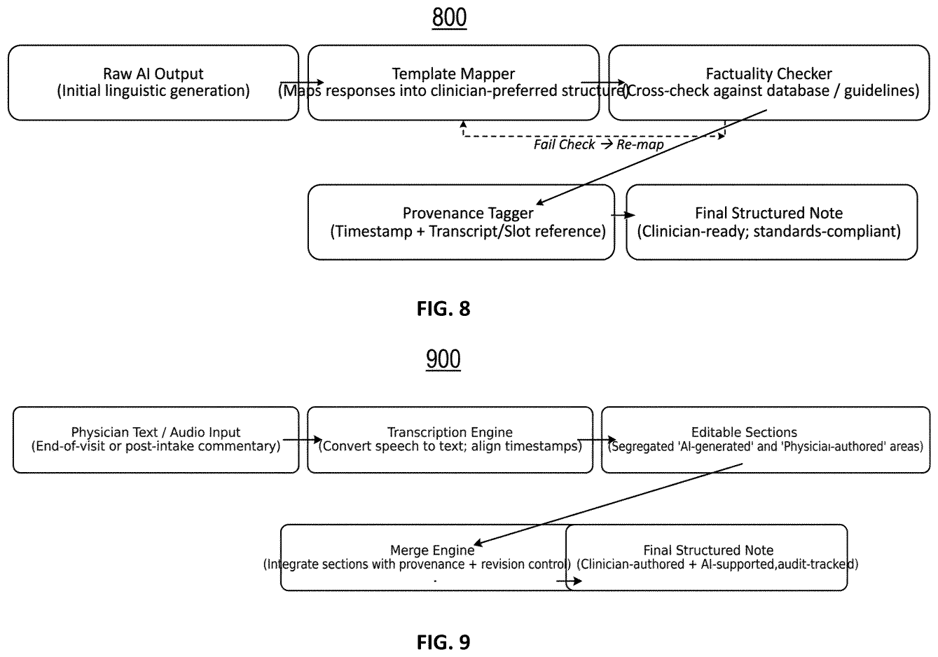

FIG. 8 is a diagram illustrating template mapping and factuality/provenance gate. The process 800 shows structured note validation and traceable provenance tagging. The process is initiated from Raw AI Output→Template Mapper→Factuality Checker (cross-check vs. clinical database/guideline)→Provenance Tag (timestamp+source)→Final Structured Note.

FIG. 9 is a diagram illustrating a clinician co-authoring merge processing flowchart. The flowchart illustrates a process 900 of collaborative editing of user/AI documentation with segregation and revisional control. The process is initiated from Physician Text/Audio Input→Transcription Engine→Editable Sections→Merge Engine→Final Note. The process provides a hybrid AI-user authorship with control and accountability (i.e., non-diagnostic compliance).

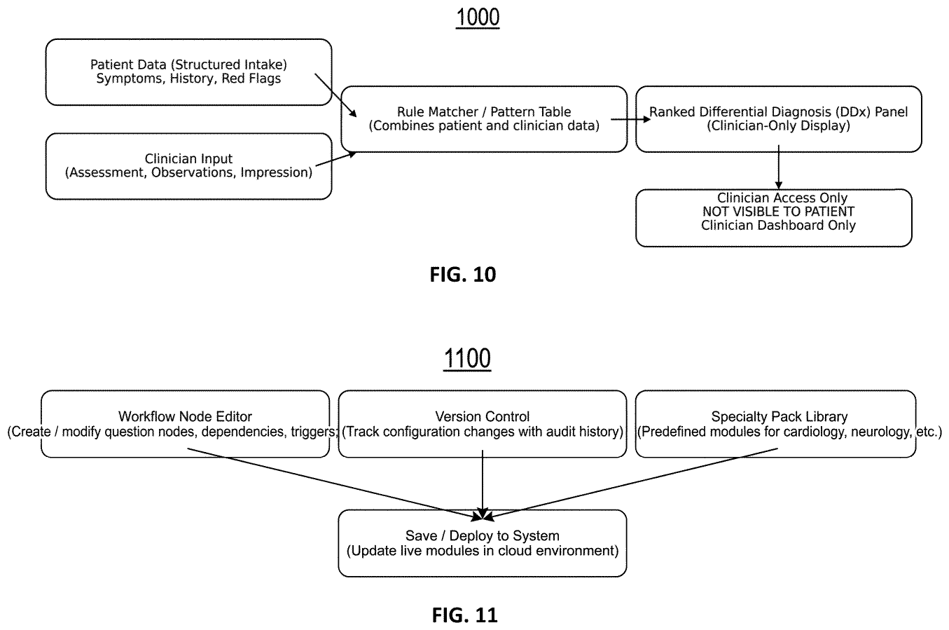

FIG. 10 is a diagram illustrating a differential diagnosis (DDx) assist processing flow chart. The flowchart illustrates a decision support process 1000 performed by the system 100 without making diagnoses. Clinician-only decision support operations are performed without generating a diagnostic output. The process is initiation from inputs: “Patient data+Clinician Input”→Rule Matcher/Pattern Table→Ranked DDx Panel (for clinician only). The ranked output is not a diagnosis and is not presented to patients.

FIG. 11 is a diagram illustrating an admin editor/configuration layer 1100 of the system. The system 100 provides an admin editor and configuration user interface. The system 100 provides functionality for workflow node editing, version control and a specialty pack library. The functionality provides modular configuration and extensibility with consideration for specialty adaptation.

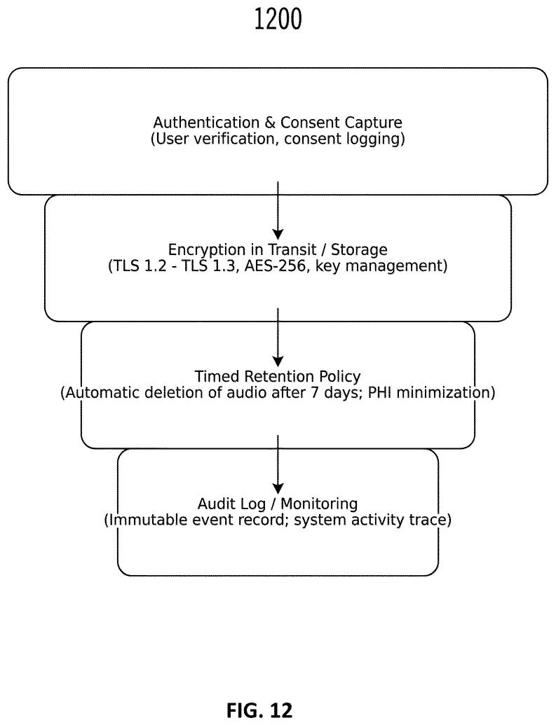

FIG. 12 is a diagram illustrating security/consent/retention control layers of the system. The system 100 provides security, content and retention functionality. The control layers 1200 comprises: Authentication & Consent→Encryption in Transit/Storage→Timed Retention (autodelete audio after 7 days)→Audit Log. These controls are configured to support compliance with HIPAA, GDPR, and applicable medical data protection standards.

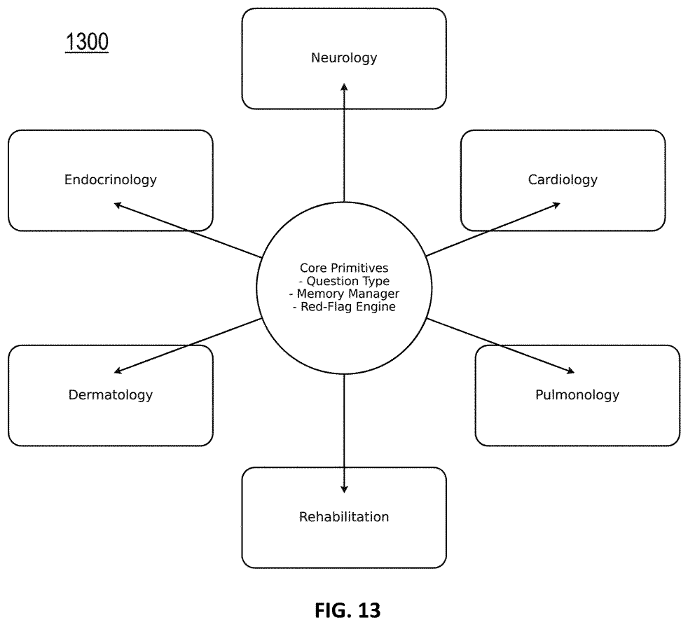

FIG. 13 is a diagram illustrating a multi-specialty overlay 1300. The system 100 is configured for multiple specialties with a core primitive. The center of the diagram illustrates core functionality performed by the system, such as question typing, memory management and red-flag processing. Different specialties may be explored by the system, such as Neurology, Cardiology, Pulmonology, Rehabilitation, etc. Different nodes or modules may be configured for one or more specialties, with each node including questions germane to the particular specialty.

### System Overview

In some embodiments, the system 100 is configured as a modular, specialty-agnostic pre-visit intake and documentation system. The system 100 executes a deterministic, adaptive workflow, via a set of predefined workflow nodes, to collect patient-reported history via telephony or web voice, generates clinician-preferred structured notes, and provides a clinician-only, non-diagnostic decision-support (DDx) addendum that the physician can revise, accept, or discard. The system 100 does not make a final diagnosis and does not provide treatment recommendations. The system 100 may be used in various domains, such as: medicine and surgical subspecialties (neurology, neuro-ophthalmology, cardiology, dermatology, etc.), rehabilitation and allied health (PT/OT/SLP), and research/coordinator intake. The decision-support addendum is not exposed to patient-facing interfaces and does not generate diagnostic determinations.

The Dynamic Workflow Controller module 158 executes specialty workflows as directed graphs of question/validation nodes. The module 158 performs branch-and-return for related topics and red-flag sub-workflows; enforces single-question cadence.

The system 100 comprises a hybrid memory system 100 that facilitates deterministic control of the specialty workflows. In some embodiments, the system 100 retains dialogue submitted by a user and may load and store in computer memory for faster processing nodes (such as asked/answered/skipped nodes); active modules; and/or a red-flag stack.

In some embodiments, the system 100 uses a workflow state manager (such as a persistent database) that includes node description, status, timestamps and override information. A per session persistent database may be used by the system 100 for each of multiple user that are interacting with the system.

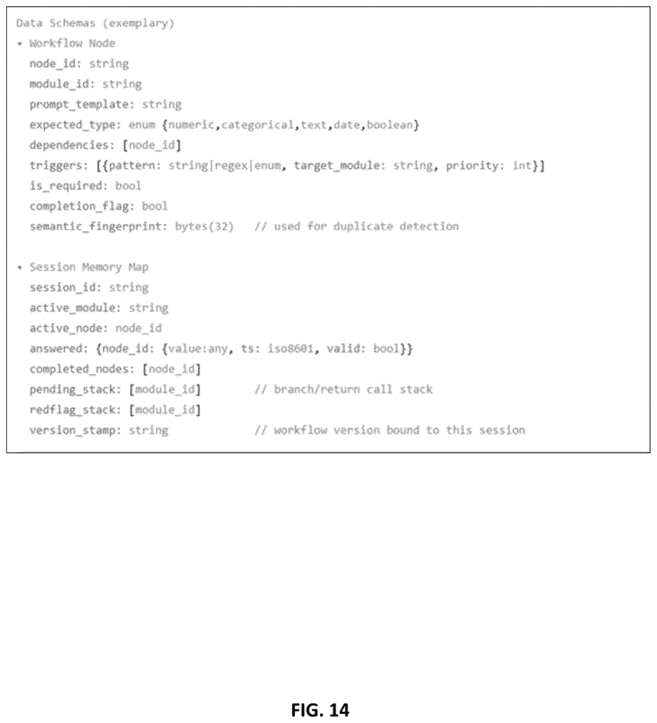

FIG. 14 is a diagram illustrating exemplary data schemas for a workflow node and a session memory map. A workflow node may include one or more identifiers for the node and module. The workflow node includes a prompt template that is used by the DWC to construct a prompt for execution by one or more LLMs. The workflow node includes an expected type of data to be collected from a user in conjunction with the prompt template. The workflow node may include one or more dependency nodes that instant node requires to have completed a priori, and/or that will be completed after the instant workflow node. The workflow node includes a definition of one or more triggers that are used by the AI agent to evaluate a response from a user. The occurrence of a trigger causes the AI agent to execute another module or other computer processes.

The session memory map is used by an AI agent to track and manage the processing of respective workflow nodes. The session memory map includes a session ID, an active module description, an active node id for a particular workflow node being processed, an answered section that identifies a workflow node and information/data submitted by a user which is then associated with the workflow node. The session memory map maintains: a listing of one or more completed workflow nodes; one or more pending stack workflow nodes for branching to another node, and return from the branched to node; one or more redflag stack workflow nodes; and a workflow version identifier.

In some embodiments, the system 100 provides a large language model (LLM) context buffer that is used for generating dialogue (such as text) while providing communication with a user. In some embodiments, the system 100 causes the LLM to perform instructions based on a generated prompt. In response to the prompt, the system 100 receives an output from the LLM. This LLM may be a lightweight, smaller sized LLM (using a reduced set of tokens) for faster processing. This smaller size LLM for example may be used for phrasing communications to a user.

In some embodiments, the system 100 generates an immutable audit log that stores information about transcript spans, branching decisions, checkpoints, physician edits, etc.

In some embodiments, the system 100 provides validation and safety processing. The system 100 may, for example, use data-type validators (numeric, categorical, date formats), dependency checks, checkpoint completion, anti-duplication (ID+semantic similarity), and PHI/consent controls.

In some embodiments, the system 100 generates documentation, transcripts, and other medical data. For example, the system 100 consumes transcript and structured slots to render a clinician-preferred template (e.g., CC, HPI, Timeline, Past Ocular/Medical, Meds, Allergies, Records Reviewed) and a patient-friendly summary. In some embodiments, the system 100 uses an LLM for linguistic normalization under factuality/provenance gating.

In some embodiments, the system 100 generates and renders for display a Clinician Co-Authoring user interface. In this user interface, a user of a Physician type may input brief text or audio after the visit (or at end of intake) of a patient to: record their impressions (e.g., “likely X; rule out Y”), specify ordered tests and instructions, request DDx assist generation constrained to patient-reported facts and the physician's stated impressions. The system 100 allows edits and modifications to the input data and includes an audit trail to track changes to the data.

In some embodiments, the system 100 generates and renders for display a Non-Diagnostic Decision-Support (DDx Assist) user interface. The user interface is configured for use by a clinician type of user. This user interface compiles evidence-linked candidates based on patient history, negatives, red-flag results, timeline, and physician input. The interface presents informational candidates only and does not perform clinical decision-making. This interface is configured strictly for review by a clinician type of user, and is not made available or accessible to a patient type of user

In some embodiments, the system 100 provides export of data and/or integration with other systems. For example, the system 100 may provide an optional FHIR/HL7 export of the clinician note; DDx panel stays in the physician user dashboard by default.

In some embodiments, the system 100 provides functionality for security and compliance. For example, the system 100 may use security/encryption functions such as TLS 1.3 in transit, AES-256 at rest; role-based access; consent ledger; PHI minimization and retention controls.

### Dynamic Workflow Controller

As discussed above, the system 100 includes a Dynamic Workflow Controller (DWC). The DWC is configured to perform one or more operations of:
(1) Primary-to-Related Branching: e.g., “vision change” module activates “diplopia” or “headache” sub-modules when triggers appear; returns to the invoking module after branch completion.
(2) Red-Flag Branching: rule table maps patterns to urgent follow-ups (e.g., nocturnal worsening, pulsatile tinnitus, acute focal deficits). The DWC must resolve red-flag branches before completing the segment.
(3) Checkpoint Processing: each segment (e.g., HPI core, ROS slice), the system 100 declares required fields, conditional fields, red flag closures; advancement is blocked until a value checkpoint is true.
(4) Anti-Duplication Processing: the system 100 may skip re-asks using node IDs+semantic matching; overlapping modules share fulfilled fields (asked once, credited to both).

In some embodiments, the system 100 generates a directed acyclic graph (DAGs) of question nodes, validation nodes, and branch edge nodes. The respective nodes each have a predefined description of one or more of the following items: a prompt template (e.g., LLM-facing phrasing to be provided to an LLM), and an expected type of data (such as, numeric, categorical, free text, date), a listing of one or more dependencies nodes (other nodes that are a pre-requisite to the instant node), a listing of one or more triggers (e.g., keywords/patterns→branches), and a checkpoint descriptor (such as a Boolean value which marks end-of-segment validation).

In some embodiments, the system 100 also provides one or more specialty modules (e.g., headache, diplopia, vision loss) which may cause the system 100 to execute related workflows (e.g., sleep disturbance, visual symptom) when semantic triggers appear. The DWC returns to the invoking module after the branch segment completes (stack-like behavior recorded in session memory).

In some embodiments, the system 100 provides a red-flag engine. The system 100 uses a rule table that maps symptoms or patterns to red-flag sub-workflows with higher priority. For example, the following shows a dynamic control rule that causes (if either criterion is true) then, which causes the system 100 to branch and execute a particular preidentified node.

In some embodiments, the system 100 performs operational control checks, such as the following: (1) type/range validation: durations→ISO-8601 or controlled vocabulary; dates→MM/YYYY or DD/MM/YYYY as required; (2) semantic validation: “double vision”→enforce monocular vs binocular follow-up; and (3) progress integrity: cannot proceed to “ROS” until “HPI core” checkpoint passes wherein red-flag processing results in workflow routing and informational indicators for clinician review rather than automated medical action.

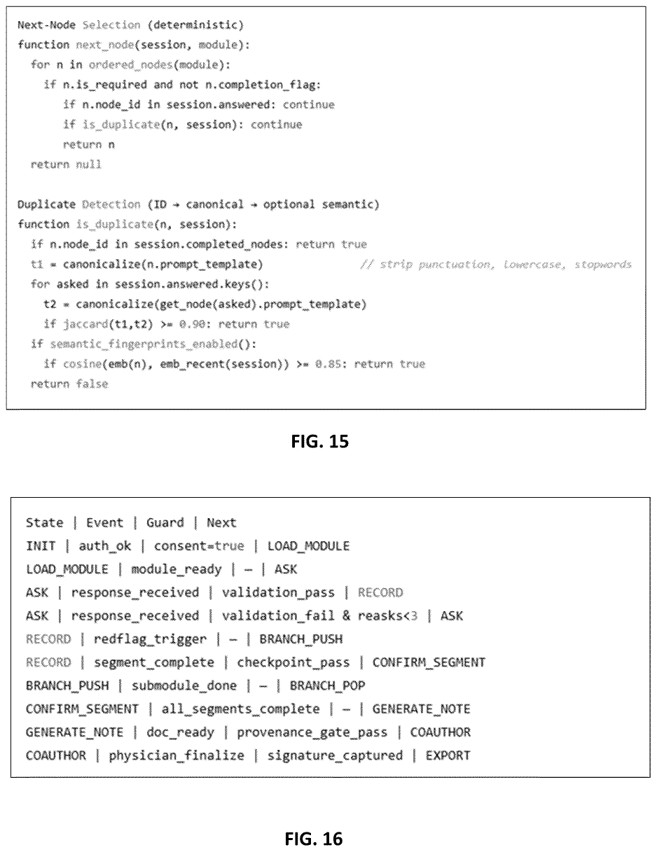

FIG. 15 is a diagram illustrating next node selection and anti-duplication of workflow nodes. The diagram illustrates processing logic for determining a next node to perform by the AI agent. Also, the diagram illustrates logic for determining potential duplicate workflow nodes and ensuring that a duplicate workflow node is not performed by the DWC.

FIG. 16 is a diagram illustrating an example transition table of a session state machine. In some embodiments, the system 100 has a deterministic, schema-based memory architecture. For example, the DWC determines what action to perform and not an LLM. For example, the DWC is configured to determine which node to execute next.

In some embodiments, the system 100 creates an operative session map. The following list illustrates parameters that may be used for the session map. Here, the system 100 created a session map for a current session (e.g., S123) and describes particular nodes being executed or activated for the current session).
{“session_id”:“123”,
“specialty_module”:“ardiology_chest_pain”,
“active_node”:“PI.Q4”,
“nodes_asked”:[“HPI.Q1”,“PI.Q2”,“PI.Q3”],
“responses”:{“HPI.Q1”:{“value”:“udden”,“ype”:“nset”}},
“completed_nodes”:[“HPI.Q1”,“PI.Q2”],
“red_flags”:[“exertional_pain”],
“stack”:[“redflag_exertional”],
“timestamp”:“025-10-27T09:42:00Z”}

In some embodiments, the system 100 receives intake (such as user input text or speech signals which are converted to text the system 100 creates a transcript of the received user input. A document generator then uses the transcript to produce reports and/or data.

In an ingestion and provenance step, the system 100 receives input such as: final transcript; structured slots; negatives; red-flag outcomes; optional external artifacts (clinic sheet OCR). Every sentence in the note links to a provenance pointer (transcript span or slot ID). Unprovenanced tokens are rejected.

In a template mapping step, required and optional sections are set according to a template specification. Missing sections from the template specification are omitted. The system 100 maps lay terminology to standardized medical terminology while preserving the original patient phrasing with provenance tags.

In some embodiments, the system 100 applies a time handling policy. In a default policy (verbatim-only), the system 100 renders dates exactly as reported, with provenance tags. In a configurable mode, the system 100 renders verbatim and normalized (approx.) month/year, which are stored with provenance.

In some embodiments, the system 100 provides report or information in multiple formats and via one or more communication channels. For example, the system 100 may generate a Clinician Note (standards-ready, with an optional FHIR Composition), and a Patient-Friendly Summary (which includes a description of no new facts and uses plain language (and not medical jargon or terminology).

In some embodiments, the system 100 applied rules for data types and validation of data types. The system 100 uses these rules to determine a “valid response” for a node requesting certain type of information from a patient user. For example, the system 100 may apply rules for: (1) Numeric: duration in minutes/hours/days; severity on 0-10 scale (range bound); (2) Categorical: onset∈{sudden, gradual}; axis∈{horizontal, vertical}; (3) Boolean: yes/no flags (e.g., nocturnal awakening); (4) Text/NL: free text summarized into slots+saved verbatim in audit trail; and (5) Date formats: enforce MM/YYYY or DD/MM/YYYY parsing; store ISO timestamps alongside.

In some embodiments, the system 100 determines an invalid or ambiguous inputs and trigger an auto re-ask with guidance (“I can record month and year, like 02/2025.”).

### System Processing Overview

The system 100 renders for display, via a user interface, to a user of a patient type consents and disclaimers and receives a user input of acceptance and stores in a database a consent record. The disclaimers clarify that the system provides documentation and workflow support and does not replace clinician judgment.

The system 100 then performs an authentication process with confirmation information received by the system 100 (such as the date of birth of the user and/or a code received by the user).

The system 100 then performs a process to identify an open-ended chief complaint (LLM narrows terms; the DWC extracts an initial topic). The system 100 receives the user input and generates a prompt for execution by an LLM. The LLM returns a response with a narrowed set of terms where the DWC extracts an initial topic for the chief complaint. The narrowed set of terms are used by the system 100 to then identify a primary module or node to execute.

The system 100 selects a primary module or node to execute for process where the primary node is associated with a topic (e.g., diplopia, vision loss, headache). The system 100 then may load into memory into a graph memory structure the other nodes that are associated with the primary node (such as child nodes, branching or called nodes).

The system 100 then generates a series of iterative questioning (single-question cadence, no multi-question prompts) based on a pre-determined set of questions associated with an instant node (e.g., here the primary node) that is being executed. The system 100 receives user input in the form of textual or spoken audio signals from the user, via a user interface, that are responsive to a respective asked question.

The system 100 evaluates the response input, optionally then branches to related or red-flag workflows as triggers arise based on the text or context of the response input.

The system 100 then confirms and/or provides an output to the user via the user interface, and in some instances asked for corrections and/or confirmations of the information provides by the user. The system 100 then makes any corrections as needed to the data provided by the user.

In some embodiments, the system 100 generates dual-format documents (such as FHIR/HL7 clinician notes and a patient-friendly summary).

During the interaction of the system 100 with the user, the system 100 performs security and audit procedures to maintain the integrity and confidentiality of information being provided by the user (e.g., encryption, access control, immutable log, etc.).

### Example of Processing a “Headache” Response

The example illustrates functionality for a Headache node or module being performed by the AI agent. The AI agent determines a red-flag occurrence by evaluating input provided by a user which causes the AI agent to branch and return. An example Headache node or module (e.g., HPI. core) may be defined as follows: (1) Required information to gather: onset (sudden/gradual), duration, frequency, quality, severity, location, exacerbating/relief factors; (2) Conditional information to gather: photophobia/phonophobia, nausea/vomiting, positional change; and (3) Trigger watchlist: awakens from sleep, whooshing tinnitus, transient vision loss, jaw claudication, scalp tenderness, neuro deficit.

Branching for an IIH/ICP Red-Flag may be defined as follows: (1) Ask: nocturnal worsening; pulsatile tinnitus; transient visual obscurations; diplopia; papilledema history; recent weight gain; OCP use; and (2) Close when all red-flag follow-ups validated→return to Headache. HPI. core at last checkpoint.

A Checkpoint may be defined where the Headache segment completes and then advances to ROS (neuro-ophth) module and then to a Past Medical/Risk Factors module.

An example Diplopia and Vision-Loss Modules may be defined where questions for this module may include questions about: monocular vs binocular splitter (closing one eye test), axis classifier (horizontal vs vertical separation), fatigability path (myasthenia), pain/optic neuropathy path (pain on eye movement; color desaturation), and stroke screen (weakness, numbness, dysarthria).

Each module contains completion checkpoints, preventing premature progression.

The system 100 provides functionality for physician co-authoring and finalization. In some embodiments, the system 100 provides for a physician input capture. After a visit (or at end of intake), a physician user adds brief text or audio (ASR transcribed) containing their impressions, exam highlights, and intended tests/instructions. In some embodiments, the system 100 provides for co-authoring merge where the system 100 segregates patient-reported data from physician-authored content and renders the final note with labeled sections (e.g., “Physician Assessment Input (verbatim)”). Physician content never retroactively changes patient statements; it is additive and clearly attributed.

The system 100 provides for decision support/differential diagnosis assist (with physician seed). On request, the differential diagnosis (DDx) module uses only (a) patient-reported facts/negatives/flags and (b) the physician's explicit input to assemble an evidence-linked candidate list and data-gap prompts. For example: “Consider idiopathic intracranial hypertension—supported by: nocturnal headache; pulsatile tinnitus; transient visual obscurations; physician observed papilledema.” No final diagnosis is generated; the physician finalizes the plan. Output remains editable and contained to dashboard by default (not exported to EHR unless configured).

In some embodiments, a patient instruction summary is automatically generated. For example, if a physician lists tests/referrals/instructions, the system 100 can produce a patient-safe handout (plain language) mirroring exactly the physician inputs—no added advice.

In some embodiments, the system 100 provides multi-specialty modules and rehabilitation coverage. Specialty modules may include modules covering topics of neurology, neuro-ophthalmology, cardiology, dermatology, endocrinology, orthopedics, etc. The system 100 may provide templates for allied health/rehabilitation such as PT/OT/SLP templates with domain-specific red-flags (e.g., new neurological deficits, red-flag back pain, post-op warning signs). The system 100 provides an administrator editor which allows clinicians to configure workflows (add/remove modules, set thresholds, change templates); all versions are tracked.

In some embodiments, in a research/coordinator mode the system 100 allows for pre-visit research intake which uses study-configured workflows (eligibility criteria, adverse-event red flags, visit windows). The system 100 outputs/populates study forms or CRF-like structures; still non-diagnostic and segregated from clinical DDx.

In some embodiments, the system 100 provides functionality for security, compliance, and retention. System 100 provides encryption using TLS 1.3/AES-256 and key management per policy. System 100 provides access control to the system 100 based on user roles (e.g., a patient user, clinician user, admin user and a coordinator user). System 100 provides a consent ledger which stores immutable records of consent, data sharing, and export of data from the system. System 100 provides PHI minimization with configurable retention periods for transcripts/audio data related to user input. System 100 does not provide any automated diagnosis or treatment. System 100 is configured for DDx is clinician-only and advisory.

The system 100 provides many technical improvements. For example, the system 100 uses deterministic memory and de-duplication which reduces the number of needed LLM executions/calls and provides reduced latency, and lower token/computational load. System 100 provides checkpointed completeness which provides higher data integrity vs. static forms. System 100 provides branch-and-return orchestration which covers red-flags without losing context. System 100 provides overlapping-segment fusion which allows for asking a question once, which satisfies multiple modules (e.g., nodes) that request the same information.

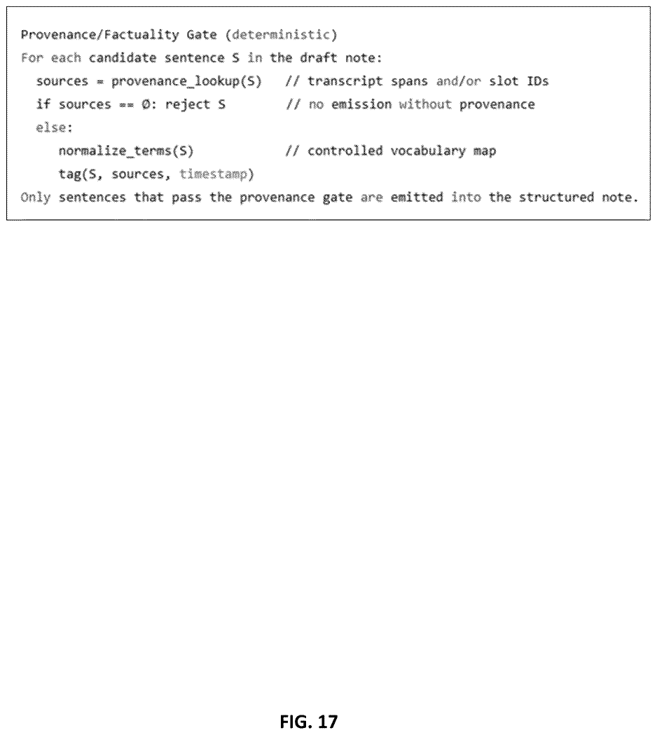

FIG. 17 is a diagram illustrating a provenance/factuality gate. System 100 provides for factuality/provenance-gated generation which prevents hallucinated content being generated by an LLM, which yields an EHR-ready structure. System 100 provides for clinician co-authoring with separation of sources which provides for safe, auditable finalization without mixing patient and physician statements. System 100 provides measurable reductions in interaction time and missing-field rates and produces standards-formatted outputs (FHIR/HL7) with higher structural integrity than static intakes.

In some embodiments, the system 100 provides an administrator/configuration user interface and functionality. The system 100 provides functionality for a workflow editor that allows an administrator usable functionality to create workflow nodes, dependencies, triggers, red-flag sets. The functionality allows for parameterization of expected types, priors (e.g., risk weights) and escalation policies. System 100 provides module versions tied to sessions and for audit controls. System 100 provides for a hierarchy of templates based on local and/or global templates. System 100 provides for per-physician user overrides.

In some embodiments, system 100 provides communication and authentication protocols. For outbound and inbound voice calls, the system 100 is configured to initiate voice calls within a customized predetermined time frame (e.g., 24-48 hours before an appointment) and to accept inbound calls from patients or authorized caregivers via both traditional telephony and web-based interfaces. For patient authentication, during a call setup, the system 100 requires that a user of a patient type must verify their identity using predetermined methods such as entering a unique code or their date of birth. This ensures that only authenticated individuals proceed with the adaptive questioning.

In some embodiments, the system 100 provides an adaptive LLM questioning workflow. The system 100 collects information from a patient of an initial symptom. The AI agent begins by asking the patient to describe the reason for the visit and most concerning symptoms relevant to the specific clinical specialty they have the appointment with.

In some embodiments, the system 100 asks questions to the patient with guided inquiry based on clinical guidelines. The system 100 follows a questioning process established clinical guidelines and ICD-10 criteria. An AI agent asks targeted questions to the patient regarding the patient's main complaints and other associated symptoms. Also, in some embodiments, the AI agent also reviews data from medical system 100 related to the medical specialty to ensure comprehensive symptom check for patients. Based on the reported symptoms, the AI agent asks additional questions to collect and structure symptom information relevant to clinician assessment of conditions with similar symptoms. The AI agent assesses aspects such as symptoms' frequency, quality, severity, duration and associated red flags.

In some embodiments, the system 100 makes dynamic adjustments and performs data integration processes. Based on the responses received, the AI agent dynamically refines subsequent questions to address any gaps in the patient's history. Once the interrogation is complete, the system 100 automatically generates a pre-visit clinic note using a specialty-specific template and reads all the collected information to patients to obtain their confirmation that all the collected data is accurate and reflects patients reported symptoms, and allows patients to make revision, corrections or add additional information if they would like to add more symptoms. After final verification by patients, AI agent generates the summary of the information in the format of a clinic note and includes a preliminary, non-diagnostic differential diagnosis list for physician review (as clinician's decision support) for reviewing and verifying the information with patients at the time of appointment, if needed.

In some embodiments, system 100 provides structured documentation and decision support. System 100 provides functionality for clinical note generation. A documentation generator compiles the gathered data into a structured note that includes sections for HPI, ROS, medication/allergy lists, and past medical history. The note is formatted according to industry standards (HL7, FHIR) for seamless integration with EHR systems.

In some embodiments, the system 100 provides preliminary decision support. The system 100 flags critical responses (such as red flags in headache presentations) and provides a preliminary, non-diagnostic list of potential differential diagnoses for clinician's review. This support is intended to assist clinicians during the patient visit and is not a substitute for clinical judgment.

In some embodiments, the system 100 may be configured in accordance with documentation, intake, and clinician-support workflows. For example, the system 100 may be implemented in a manner consistent with documentation-focused operational use. The system 100 in certain implementations is configured for the collection of patient-reported symptoms and medical history taking, and for the generation of preliminary decision support. It may operate primarily on conversational and patient-reported information and function as an assistive documentation and workflow-support platform rather than an autonomous diagnostic or treatment-generating system. The system 100 is excluded from FDA regulation because the system 100 functions exclusively as a data collection and documentation tool. The system 100 may in some implementations be deployed in non-diagnostic documentation-support environments. In some embodiments, an AI agent is prompt-engineered on the basis of current medical literature and clinical guidelines in different medical specialties (such as internal medicine, neurology, cardiology, etc.). For instance, for a patient referred to a neurology clinic with complaints of headaches, the system 100 begins by asking the patient to describe the reason for the upcoming visit and their concerning symptoms. System 100 then employs a series of specialty-specific prompts to gather detailed information on headache characteristics (frequency, severity, quality, duration) and associated symptoms. System 100 uses established headache guidelines and ICD-10 criteria to collect symptom patterns associated with clinically recognized headache categories for clinician review and asks additional questions to identify red flags (such as associated visual disturbances, double vision, nocturnal headaches, etc.). The system 100 then generates follow-up queries to capture additional patient history including known diagnoses and current medications.

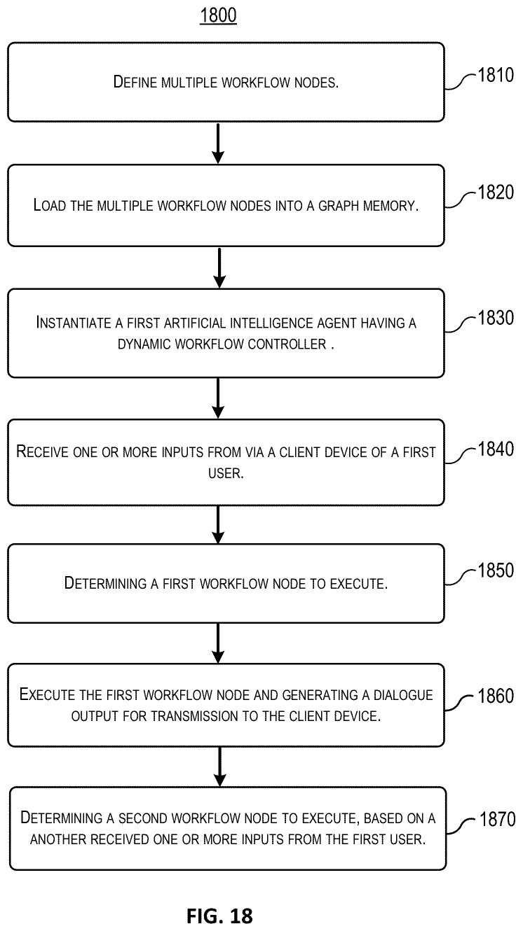

FIG. 18 is a flow chart illustrating an exemplary method 1800 that may be performed in some embodiments. In step 1810, the system 100 defines multiple workflow nodes (also referred to a workflow module). The multiple workflow nodes include questions to be answered by a user and/or required data to be received from a user.

In step 1820, the system 100 loads into a memory of a computer system, the defined multiple workflow nodes into a memory graph.

In step 1830, the system 100 executes (e.g., instantiates) a first artificial intelligence (AI) agent comprising a session memory map and a dynamic workflow controller (DWC) communicatively coupled to one or more large language models (LLMs).

In step 1840, the system 100 receives a first set of one or more inputs from a first user, via a client device, wherein the one or more inputs comprise voice signals and/or textual input, and the system 100 stores in the session memory map of the first AI agent, the received first set of one or more inputs.

In step 1850, the system 100 determines, via the AI agent, based on the received first set of one or more inputs, a first workflow node to execute. The AI agent then executes the first workflow node.

In step 1860, the system 100 perform by the DWC a workflow operation comprising: (a) executing the first workflow node, via the DWC, wherein the DWC retrieves the questions and/or required and generates a prompt for execution by the one or more LLMs; and causing the one or LLMs to execute the prompt and return an output in a form of a first question for communication to the first user; and (b) performing an LLM hallucination validation to determine whether the LLM generated a hallucinated output, and corrects the output to a non-hallucinated form. The system 100 transmits to the client device, the form of the question as an audio signal or text.

In step 1870, the system 100 receives a second set of one or more inputs from a first user, via a client device, wherein the one or more inputs comprise voice signals and/or textual input, and storing in the session memory of the first AI agent the received second set of one or more inputs. The system 100 determines, via the first AI agent, based on the received one or more inputs, a second workflow node to execute. The AI agent then executes the second workflow node.

In some embodiments, the AI Agent may branch into a red-flag sub-workflow node based on detected trigger patterns detected in the received first set of one or more inputs. After performing the operations of the red-flag sub-workflow node, then execution of the first workflow node is resumed upon completion of processing the red-flag sub-workflow node.

In some embodiments, the AI Agent, via the DWC, performs a workflow operation by executing the second workflow node, via the DWC, wherein the DWC retrieves the questions and/or required and generates a prompt for execution by the one or more LLMs. The DWC causes the one or LLMs to execute the prompt and return a second output in a form of a second question for communication to the first user. Then system 100 transmits to the client device, the form of the second question as an audio signal or text.

The system 100 then receives a second set of one or more inputs from the first user, via the client device, wherein the one or more inputs comprise voice signals and/or textual input. The system 100 stores in the session memory map of the first AI agent the received second set of one or more inputs. The AI Agent then determines, based on the received one or more inputs, a third workflow node to execute.

In some embodiments, the system 100 performs an LLM hallucination validation to determine whether the LLM generated a hallucinated output for the second output and corrects the output to a non-hallucinated form.

In some embodiments, the system 100 determines a set of validation checkpoints, and prevents advancement to the second workflow node until validation checkpoints are satisfied for the first workflow node.

In some embodiments, the system 100 branches into a red-flag sub-workflow node based on trigger patterns detected in the received second set of one or more inputs, and then returns to the execution of the second workflow node upon completion of processing the red-flag sub-workflow node.

In some embodiments, the system 100 determines a set of validation checkpoints, and prevents advancement to a subsequent workflow node until validation checkpoints are satisfied for the second workflow node.

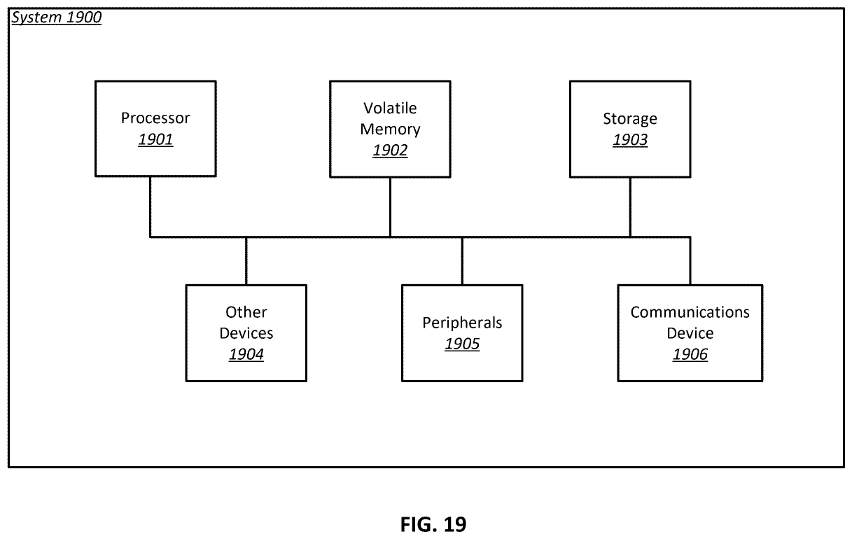

FIG. 19 is a diagram illustrating an exemplary computer that may perform processing in some embodiments. Exemplary computer 1900 may perform operations consistent with some embodiments. The architecture of computer 1900 is exemplary. Computers can be implemented in a variety of other ways. A wide variety of computers can be used in accordance with the embodiments herein.

Processor 1901 may perform computing functions such as running computer programs. The volatile memory 1902 may provide temporary storage of data for the processor 1901. RAM is one kind of volatile memory. Volatile memory typically requires power to maintain its stored information. Storage 1903 provides computer storage for data, instructions, and/or arbitrary information. Non-volatile memory, which can preserve data even when not powered and including disks and flash memory, is an example of storage. Storage 1903 may be organized as a file system, database, or in other ways. Data, instructions, and information may be loaded from storage 1903 into volatile memory 1902 for processing by the processor 1901.

The computer 1900 may include peripherals 1905. Peripherals 1805 may include input peripherals such as a keyboard, mouse, trackball, video camera, microphone, and other input devices. Peripherals 1905 may also include output devices such as a display. Peripherals 1905 may include removable media devices such as CD-R and DVD-R recorders/players. Communications device 1906 may connect the computer 1900 to an external medium. For example, communications device 1806 may take the form of a network adapter that provides communications to a network. A computer 1900 may also include a variety of other devices 1804. The various components of the computer 1900 may be connected by a connection medium such as a bus, crossbar, or network.

It will be appreciated that the present disclosure may include any one and up to all of the following examples.

Example 1: A system for adaptive pre-visit medical history collection, structured documentation generation, and clinician-only non-diagnostic decision support, comprising:
a) a communication interface configured to receive authenticated voice or web-based input from a patient through a telephony or internet protocol connection, the interface including:
a speech recognition engine configured to convert patient voice responses into text,
an optional text-to-speech engine for bidirectional interaction, and
authentication logic requiring at least one identifier selected from a patient-specific code, date of birth, or equivalent credential;
   b) a dynamic workflow controller (DWC) communicatively coupled to a large language model (LLM) and a rule-based logic engine, the DWC being configured to:
   execute a specialty-specific, directed acyclic graph (DAG) of question and validation nodes;
   select, generate, and sequence questions using anti-duplication and dependency rules;
   branch into red-flag sub-workflows based on detected trigger patterns and return to the primary module upon completion; and
   prevent advancement to subsequent modules until validation checkpoints are satisfied;
   c) a hybrid session memory system comprising:
   a session memory map stored in volatile memory tracking asked, answered, and skipped nodes;
   a persistent workflow state manager storing node status, timestamps, and physician overrides;
   a transient LLM context buffer for natural language phrasing; and
   an immutable audit log archiving conversation spans, branching events, and physician edits;
   d) a validation and safety layer configured to enforce data type validation, dependency integrity, semantic disambiguation, red-flag resolution, and duplication suppression;
   e) a documentation generator configured to:
   transform validated transcript segments and structured slots into a clinician-preferred documentation template comprising sections including at least a chief complaint, history of present illness, past medical and ocular history, medications, allergies, records reviewed, and review of systems; and
   apply factuality and provenance gating to include only sentences linked to explicit transcript spans or structured slot identifiers;
f) a clinician co-authoring interface configured to receive physician-authored text or audio input after or during the patient intake session, the interface being further configured to:
transcribe audio into text,
segregate patient-reported and physician-authored content,
render physician-authored content in a distinct labeled section, and
allow the physician to revise or append content without overwriting patient data;
g) a non-diagnostic decision-support (DDx) engine configured to:
generate a clinician-only, evidence-linked differential diagnosis list based on patient-reported facts, red-flag outcomes, and physician input,
restrict outputs to the clinician dashboard by default, and
exclude all diagnostic or treatment recommendations from patient-facing summaries; and
h) a security and compliance subsystem configured for data encryption, role-based access control, consent management, and audit retention according to applicable medical privacy standards.

Example 2. The system of Example 1, wherein the dynamic workflow controller executes specialty modules selected from a library of predefined clinical templates corresponding to neurology, cardiology, ophthalmology, dermatology, or rehabilitation medicine.

Example 3. The system of any one of Examples 1-2, wherein red-flag sub-workflows are resolved prior to continuation of the main module and present clinician-visible indicators through the dashboard.

Example 4. The system of any one of Examples 1-3, wherein the hybrid session memory system deterministically controls conversation flow independently of the LLM's probabilistic context memory.

Example 5. The system of any one of Examples 1-4, wherein the documentation generator enforces factuality and provenance gating to prevent inclusion of unverifiable statements in the clinician note.

Example 6. The system of any one of Example 1-5, wherein the physician co-authoring interface integrates a real-time transcription service and allows editable merging of AI-generated and human-authored content.

Example 7. The system of any one of Examples 1-6, wherein the non-diagnostic decision-support engine references medical guidelines and evidence tables to construct differential diagnosis candidates but does not assign likelihoods or final diagnoses.

Example 8. The system of any one of Examples 1-7, wherein the system further generates a patient-friendly summary formatted in plain language that omits physician impressions and the differential diagnosis addendum.

Example 9. The system of any one of Examples 1-8, wherein the documentation generator supports export of clinician notes via standardized protocols selected from HL7 and FHIR.

Example 10. The system of any one of Examples 1-9, wherein the system is configured to operate in a research coordinator mode that collects pre-visit research data and exports de-identified datasets for study analysis.

Example 11. A method for automated pre-visit history collection and clinician-assisted documentation, comprising the steps of:
a) receiving authenticated patient voice input via a telephony or web-based channel;
b) converting voice input to text and parsing patient statements into structured data slots;
c) executing a specialty-specific adaptive workflow including branching to related or red-flag sub-modules;
d) updating a session memory map and workflow state manager with node identifiers, completion status, and validation checkpoints;
e) detecting duplicate or redundant questions using node identifiers and semantic similarity comparison, and skipping redundant prompts;
f) upon completion of the intake, generating a structured clinician note comprising sections including at least a chief complaint, history of present illness, past medical history, medications, and review of systems, wherein each sentence in the note is linked to a provenance tag referencing the corresponding transcript span;
g) receiving optional physician input as audio or text, appending it as a distinct, labeled physician-authored section without modifying patient-reported content;
h) generating a non-diagnostic clinician-only decision support addendum based on the combined dataset of patient-reported and physician-provided information; and
i) securely storing or exporting the structured note with encryption, access control, and audit logging, wherein the decision support addendum remains within the clinician dashboard and is not exported to patient-facing documents or electronic health records unless explicitly authorized.

Example 12. The method of Example 11, further comprising enforcing completion checkpoints for each module prior to advancing to a next workflow segment.

Example 13. The method of any one of Examples 11-12, wherein all stored patient interactions are encrypted using AES-256 and all communication is protected by TLS 1.3.

Example 14. The method of any one of Examples 11-13, wherein all workflow modules are version-controlled, and the audit log records which version of a module governed an interaction.

Example 15. A non-transitory computer readable medium that stores executable program instructions that when executed by one or more computing devices configure the one or more computing devices to perform operations comprising any one of the method of Examples 11-14.

Some portions of the preceding detailed descriptions have been presented in terms of algorithms, equations and/or symbolic representations of operations on data bits within a computer memory. These algorithmic and/or equation descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general-purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. A computer-implemented method, performed by one or more computer processors, comprising the operations of:
defining multiple workflow nodes comprising questions to be answered by a user and/or required data to be received from a user;
loading into a memory of a computer system, the defined multiple workflow nodes into a directed acyclic graph (DAG) data structure, each workflow node including at least a prompt template, an expected response data type, one or more dependency identifiers, and at least one completion checkpoint;
executing a first artificial intelligence (AI) agent comprising a session memory map and a dynamic workflow controller (DWC) communicatively coupled to one or more large language models (LLMs), wherein the DWC is configured to select and sequence workflow nodes deterministically and independently of probabilistic outputs generated by the one or more LLMs;
receiving a first set of one or more inputs from a first user, via a client device, wherein the one or more inputs comprise voice signals and/or textual input, and storing in the session memory map of the first AI agent the received first set of one or more inputs;
determining, via the AI agent, based on the received first set of one or more inputs, by the DWC and based on the dependency identifiers and completion states stored in the session memory map, a first workflow node to execute;
performing by the DWC a workflow operation comprising the following:
(a) executing the first workflow node, via the DWC, wherein the DWC retrieves the questions and/or required prompt template associated with the first workflow node and generates a constrained prompt for execution by the one or more LLMs; and
causing the one or LLMs to execute the prompt and return an output in a form of a first question for communication to the first user; and
(b) validating the LLM-generated output by enforcing the expected response data type, rejecting content not traceable to the prompt template or stored user inputs, and suppressing duplicate workflow nodes using identifier-based and semantic comparison;
transmitting to the client device, the form of the question as an audio signal or text;
receiving a subsequent set of one or more inputs from a first user, via a client device, wherein the one or more inputs comprise voice signals and/or textual input, and storing in the session memory of the first AI agent the received second set of one or more inputs; and
determining, via the first AI agent, based on the received one or more inputs, a second workflow node to execute only after satisfaction of the completion checkpoint associated with the first workflow node.

2. The computer-implemented method of claim 1, further comprising:
branching into a red-flag sub-workflow node based on detected trigger patterns detected in the received first set of one or more inputs; and
returning to the execution of the first workflow node upon completion of processing the red-flag sub-workflow node only after resolution of the red-flag sub-workflow node and satisfaction of a red-flag completion condition recorded in the session memory map.

3. The computer-implemented method of claim 1, further comprising:
performing by the DWC a workflow operation comprising one of the following:
executing the second workflow node, via the DWC, wherein the DWC retrieves the questions and/or prompt template associated with the second workflow node and generates a constrained prompt for execution by the one or more LLMs; and
causing the one or LLMs to execute the prompt and return a second output in a form of a second question for communication to the first user; and
transmitting to the client device, the form of the second question as an audio signal or text;
receiving a second set of one or more inputs from the first user, via the client device, wherein the one or more inputs comprise voice signals and/or textual input, and storing in the session memory map of the first AI agent the received second set of one or more inputs; and
determining, via the first AI agent, based on the received one or more inputs, a third workflow node to execute based on dependency identifiers and completion states stored in the session memory map; and
executing the third workflow node.

4. The computer-implemented method of claim 3, further comprising:
validating the second output by enforcing an expected response data type, rejecting content not traceable to stored user inputs or the associated prompt template, and recording a validation result in the session memory map.

5. The computer-implemented method of claim 1, further comprising:
determining a set of validation checkpoints; and
preventing advancement to the second workflow node until validation checkpoints are satisfied for the first workflow node as determined by Boolean completion values stored in the session memory map.

6. The computer-implemented method of claim 3, further comprising:
branching into a red-flag sub-workflow node based on trigger patterns detected in the received second set of one or more inputs; and
returning to the execution of the second workflow node upon completion of processing the red-flag sub-workflow node only after resolution of the red-flag sub-workflow node and update of a red-flag status indicator in the session memory map.

7. The computer-implemented method of claim 3, further comprising:
determining a set of validation checkpoints; and
prevent advancement to a subsequent workflow node until validation checkpoints are satisfied for the second workflow node according to dependency and completion data stored in the session memory map.

8. A non-transitory computer readable medium that stores executable program instructions that when executed by one or more computing devices configure the one or more computing devices to perform operations comprising:
defining multiple workflow nodes comprising questions to be answered by a user and/or required data to be received from a user;
loading into a memory of a computer system, the defined multiple workflow nodes into a directed acyclic graph (DAG) data structure, each workflow node including at least a prompt template, an expected response data type, one or more dependency identifiers, and at least one completion checkpoint;
executing a first artificial intelligence (AI) agent comprising a session memory map and a dynamic workflow controller (DWC) communicatively coupled to one or more large language models (LLMs) wherein the DWC is configured to select and sequence workflow nodes deterministically and independently of probabilistic outputs generated by the one or more LLMs;
receiving a first set of one or more inputs from a first user, via a client device, wherein the one or more inputs comprise voice signals and/or textual input, and storing in the session memory map of the first AI agent the received first set of one or more inputs;
determining, via the AI agent, based on the received first set of one or more inputs, by the DWC and based on dependency identifiers and completion states stored in the session memory map, a first workflow node to execute;
performing by the DWC a workflow operation comprising one of the following:
(a) executing the first workflow node, via the DWC, wherein the DWC retrieves the questions and/or required prompt template associated with the first workflow node and generates a constrained prompt for execution by the one or more LLMs; and
causing the one or LLMs to execute the prompt and return an output in a form of a first question for communication to the first user; and
(b) validating the LLM-generated output by enforcing the expected response data type, rejecting content not traceable to the prompt template or stored user inputs, and suppressing duplicate workflow nodes using identifier-based and semantic comparison;
transmitting to the client device, the form of the question as an audio signal or text;
receiving a second set of one or more inputs from a first user, via a client device, wherein the one or more inputs comprise voice signals and/or textual input, and storing in the session memory of the first AI agent the received second set of one or more inputs; and
determining, via the first AI agent, based on the received one or more inputs, a second workflow node to execute only after satisfaction of the completion checkpoint associated with the first workflow node.

9. The non-transitory computer readable medium of claim 8, further comprising:
branching into a red-flag sub-workflow node based on detected trigger patterns detected in the received first set of one or more inputs; and
returning to the execution of the first workflow node upon completion of processing the red-flag sub-workflow node after updating a red-flag completion state stored in the session memory map.

10. The non-transitory computer readable medium of claim 8, further comprising:
performing by the DWC a workflow operation comprising one of the following:
executing the second workflow node, via the DWC, wherein the DWC retrieves the questions and/or required prompt template associated with the second workflow node and generates a constrained prompt for execution by the one or more LLMs; and
causing the one or LLMs to execute the prompt and return a second output in a form of a second question for communication to the first user based on dependency identifiers and completion states stored in the session memory map; and
transmitting to the client device, the form of the second question as an audio signal or text;
receiving a second set of one or more inputs from the first user, via the client device, wherein the one or more inputs comprise voice signals and/or textual input, and storing in the session memory map of the first AI agent the received second set of one or more inputs; and
determining, via the first AI agent, based on the received one or more inputs, a third workflow node to execute; and
executing the third workflow node.

11. The non-transitory computer readable medium of claim 10, further comprising:
validating the second output by enforcing an expected response data type, rejecting content not traceable to stored user inputs or an associated prompt template, and recording a validation result in the session memory map.

12. The computer-implemented method of claim 1, further comprising:
determining a set of validation checkpoints; and
preventing advancement to the second workflow node until validation checkpoints are satisfied for the first workflow node as determined by Boolean completion values stored in the session memory map.

13. The non-transitory computer readable medium of claim 10, further comprising:
branching into a red-flag sub-workflow node based on a trigger patterns detected in the received second set of one or more inputs; and
returning to the execution of the second workflow node upon completion of processing the red-flag sub-workflow node after updating a red-flag completion state stored in the session memory map.

14. The non-transitory computer readable medium of claim 10, further comprising:
determining a set of validation checkpoints; and
prevent advancement to a subsequent workflow node until validation checkpoints are satisfied for the second workflow node according to dependency identifiers and completion states stored in the session memory map.

15. A system comprising one or more processors configured to perform the operations of:
defining multiple workflow nodes comprising questions to be answered by a user and/or required data to be received from a user;
loading into a memory of a computer system, the defined multiple workflow nodes into a directed acyclic graph (DAG) data structure, each workflow node including at least a prompt template, an expected response data type, one or more dependency identifiers, and at least one completion checkpoint;
executing a first artificial intelligence (AI) agent comprising a session memory map and a dynamic workflow controller (DWC) communicatively coupled to one or more large language models (LLMs) wherein the DWC is configured to select and sequence workflow nodes deterministically and independently of probabilistic outputs generated by the one or more LLMs;
receiving a first set of one or more inputs from a first user, via a client device, wherein the one or more inputs comprise voice signals and/or textual input, and storing in the session memory map of the first AI agent the received first set of one or more inputs;
determining, via the AI agent, based on the received first set of one or more inputs, by the DWC and based on dependency identifiers and completion states stored in the session memory map, a first workflow node to execute;
performing by the DWC a workflow operation comprising one of the following:
(a) executing the first workflow node, via the DWC, wherein the DWC retrieves the questions and/or required prompt template associated with the first workflow node and generates a constrained prompt for execution by the one or more LLMs; and
causing the one or LLMs to execute the prompt and return an output in a form of a first question for communication to the first user; and
(b) validating the LLM-generated output by enforcing the expected response data type, rejecting content not traceable to the prompt template or stored user inputs, and suppressing duplicate workflow nodes;
transmitting to the client device, the form of the question as an audio signal or text;
receiving a second set of one or more inputs from a first user, via a client device, wherein the one or more inputs comprise voice signals and/or textual input, and storing in the session memory of the first AI agent the received second set of one or more inputs; and
determining, via the first AI agent, based on the received one or more inputs, a second workflow node to execute only after satisfaction of the completion checkpoint associated with the first workflow node.

16. The system of claim 15, further comprising:
branching into a red-flag sub-workflow node based on detected trigger patterns detected in the received first set of one or more inputs; and
returning to the execution of the first workflow node upon completion of processing the red-flag sub-workflow node after update of a red-flag completion state stored in the session memory map.

17. The system of claim 15, further comprising:
performing by the DWC a workflow operation comprising one of the following:
executing the second workflow node, via the DWC, wherein the DWC retrieves the questions and/or required prompt template associated with the second workflow node and generates a constrained prompt for execution by the one or more LLMs; and
causing the one or LLMs to execute the prompt and return a second output in a form of a second question for communication to the first user; and
transmitting to the client device, the form of the second question as an audio signal or text;
receiving a second set of one or more inputs from the first user, via the client device, wherein the one or more inputs comprise voice signals and/or textual input, and storing in the session memory map of the first AI agent the received second set of one or more inputs; and
determining, via the first AI agent, based on the received one or more inputs, a third workflow node to execute based on dependency identifiers and completion states stored in the session memory map; and
executing the third workflow node.

18. The system of claim 17, further comprising:
validating the second output by enforcing an expected response data type and rejecting content not traceable to stored user inputs or an associated prompt template.

19. The system of claim 15, further comprising:
determining a set of validation checkpoints; and
preventing advancement to the second workflow node until validation checkpoints are satisfied for the first workflow node as determined by completion values stored in the session memory map.

20. The system of claim 17, further comprising:
branching into a red-flag sub-workflow node based on trigger patterns detected in the received second set of one or more inputs; and
returning to the execution of the second workflow node upon completion of processing the red-flag sub-workflow node after update of a red-flag status indicator stored in the session memory map.

21. The system of claim 17, further comprising:
determining a set of validation checkpoints; and
prevent advancement to a subsequent workflow node until validation checkpoints are satisfied for the second workflow node according to dependency identifiers and completion states stored in the session memory map.

22. The computer-implemented method of claim 1, further comprising:
executing, by the dynamic workflow controller, a plurality of LLM execution passes, each execution pass being associated with a distinct functional role and a distinct controller state, the plurality of execution passes comprising:
(a) a first execution pass configured to generate a draft summary output from a transcript based on a clinician-specific preference profile;
(b) a second execution pass configured to review the draft summary output for factual accuracy and language compliance and to generate a corrected summary output;
(c) a third execution pass configured to extract structured identifiers from at least one of the transcript or the corrected summary output and store the structured identifiers separately from narrative content; and
(d) a fourth execution pass configured to analyze the transcript and one or more prior outputs to generate retrospective quality metrics and improvement signals;
wherein advancement between execution passes is controlled by the dynamic workflow controller and is conditioned on satisfaction of pass-specific validation criteria.
